(19) United States
(12) Patent Application Publication
(10) Pub. No.: US 2023/0051238 A1
(43) Pub. Date: Feb. 16, 2023

(54) A CLIP TO STOP NOSEBLEED

(71) Applicant: Ahmet ISLAM, Istanbul (TR)

(72) Inventor: Ahmet ISLAM, Istanbul (TR)

(21) Appl. No.: 17/794,456

(22) PCT Filed: Jan. 21, 2020
(86) PCT No.: PCT/TR2020/050041

§ 371 Date: Jul. 21, 2022

(51) Int. Cl. A61B 17/122; A61B 17/24

(52) U.S. Cl. CPC A61B 17/1227; A61B 17/24; A61B 2017/12004

(57) ABSTRACT

Disclosed is a biocompatible hemostatic nose clip having an interconnected body, arms and hands, which is intended for use in nosebleeds (epistaxis) that originate from the nasal septum, and is suitable for the personal use of children or adults without the need for medical personnel. The nose clip is insertable or removable with one hand.

### TECHNICAL FIELD

The invention relates to medical apparatus for stopping nosebleeds.

In particular, the invention relates to a biocompatible hemostatic nose clip which is intended for use in nosebleeds that originate from nasal septum (epistaxis), suitable for the personal use of children or adults without the need for medical personnel, insertable or removable with one-hand.

### STATE OF ART

Nosebleeds (epistaxis) are common in the community (including adults and children). 60 out of 100 people suffer from nosebleeds at some time in their lives. Although there are local and systemic causes of nosebleeds, some of them are idiopathic and the causes cannot be found. 90-95% of nosebleeds, including adults and children, are bleedings from the anterior part of the nose called “anterior epistaxis”. The anterior epistaxis originates from the vascular network called “Kiesselbach plexus” in the anterior part of the nasal septum called “Little's area”. The Little's area is about 1 cm away from the nasal inlet and Includes an area of 1-1.5 cm in diameter.

6-10% of people who suffer from nosebleeds admit to health institutions (emergency department, ENT, etc.). Nosebleeds may occur at different times (day-night) and away from health institutions. Therefore, it is often frightening (troublesome) for the person. Among the personal interventions to stop bleeding in the anterior epistaxis, the most common applications are to squeeze the anterior part of the nose by fingers or manually cushion it with cotton products to completely obstruct the nostrils. Other practices than those, require a health institution and a health professional. Applying pressure to the nose while going to the health institution is difficult for the person experiencing a nosebleed. Applying pressure to the nose disables the hand by having to keep one hand constantly on the nose and makes it impossible to breathe through the nose as the nose is completely blocked. In addition, if the pressure applied to the nose does not coincide with the bleeding vessel, the bleeding does not stop and flows back into the throat and nasopharynx of the patient/person.

Apart from the applications of health institutions and healthcare professionals, medical apparatuses under the names of clips, clamps, pegs, splints, etc., have been developed, which provide compression of the nose externally to stop nosebleeds. These existing apparatuses essentially have the same function as squeezing the nose with fingers of a person. In cases where these apparatuses are used, the nose is completely obstructed, and the user cannot breathe through the nose. In addition, if the apparatus used cannot compress the bleeding vessel, the nasal bleeding continues into the nasopharynx and throat.

For example; patent application GB2363575A relates to a nasal clip applied externally to stop an internal nosebleed. It comprises two pads connected to each other by a flexible connecting piece arranged to press the pads towards each other to hold the patient's nose externally. The pads are preferably waterproof. In use, the nose clip is placed on the outer part of the nose of a patient in the Little's area and serves to stop internal nosebleed. However, the mentioned clip closes the nose by external pressure and obstructs breathing through the nose. Furthermore, since it should be placed in the Little's area, it is unlikely that the correct application will be provided for personal use.

A similar structure described in patent application U.S. Pat. No. 5,899,918A is also related to a nasal compression device used externally to stop anterior nosebleeds. This device acts like a latch. It is placed on the nose externally to fit the soft compressible portions of the nose side walls by the user. As seen in this example, existing apparatuses applied externally to the nose, while exerting external pressure on the nose, may not exert pressure on the entire Little's area where nasal bleeding is most common; and the nosebleed may continue. However, by compressing the nose externally the nasal airway is completely obstructed, and long-term use is not suitable as the patient is disabled from breathing through the nose.

When the medical devices applied into the nose are examined, it is seen that nasal ventilation clips and anti-snoring clips are used to open the airway by opening the nostrils and to increase the nasal breathing. These clips are not intended to stop nosebleeds, and they can cause nosebleeds by causing possible irritation of the nasal mucosa.

For example, a national utility model application TR2008/01050 discloses an apparatus for relaxing breathing. This developed product is a nose clip consisting of two arms connected to each other in a V-shape with between 110-120 degrees. In practice, the user places the clip with the arms in the nose, the angled arms pushes the side walls of the nose outwards to help keep the airway open. Due to this mechanical effect of the invention, it is claimed to aid nasal breathing in conditions such as common cold, influenza or snoring. However, it is clearly not suitable for use in nosebleeds, nor can any variation be adapted as hemostatic.

In present applications, medical apparatuses, such as pencet, clamps, forceps, which are connected with sponges or absorbent articles in the ends, and are specifically designed for the use of healthcare personnel, have been used to exert pressure in the nose to stop bleeding. These apparatuses are generally constructed on a horizontal plane comprising holding areas remaining outside the nose, extensions that penetrate to the nostrils, and a sponge or absorbent material positioned at end of the extensions corresponding to the bleeding site. In order to shorten the bleeding stop period, these sponges or absorbents are immersed in pharmaceuticals such as hemostatic drugs and placed in the bleeding site before administration. Since these products are not suitable to stay in the nose for a long time, it is aimed to use the chemical effect of pharmaceuticals together with mechanical pressure effect in stopping bleeding.

Since these products for stopping internal bleeding in the nose are constructed in one-directional (horizontal), the holding and/or application areas remaining outside of the nose close the upper lip of the patient; so, cause difficulty for patient in speaking, eating and drinking. The extensions entering the nostrils are not large enough to create pressure, and do not cover entire of the Little's area. This results in the necessity of bringing the ends along to the Little's area for the user. The sponges or pads connected to the pressure end obstruct the nose by swelling secondary to the impregnated pharmaceuticals, blood and nasal discharge during administration, and are not suitable for prolonged use because it makes breathing difficult. Since the areas other than sponges or pads in these products are usually made of materials such as metal, they are not biocompatible and irritate the mucosa. Although it has been described that these existing products are suitable for personal use, due to all mentioned disadvantages, the present products are not successful in personal use.

For example, patent application CN104758026A relates to a disposable hemostatic clip. The clip of the present invention comprises two interconnected forceps and discs at the end of these forceps, the distance between which is adjusted by a stud and nut. The mentioned clip aims to keep the airway open while stopping bleeding by creating pressure on the nasal septum. The invention is a disposable apparatus developed for the application of healthcare personnel. In practice, a healthcare professional should detect the bleeding site in the nasal septum and place the Vaseline gauze layer and discs in order to lubricate the mucosa, and provide the optimum pressure with the help of the nut, avoiding excessive compression that will cause necrosis. However, this procedure can only be performed by a healthcare professional, and it is not suitable for personal use by patients.

In the patent application TR 2016/14678, the invention relates to removable nasal tampon for quicker recovery of swelling and edema after nasal surgery. Said bumper configuration consists of pressure bars connected by a pair of triangular hollow pads in the nose air channel, which allow breathing through the nose, and clamping system for opening and closing these pressure bars outside the nose. Since the entire structure is connected in a horizontal plane, the clamp portion outside the nose will close the upper lip and cause discomfort in the patient's speaking and eating-drinking functions. The mentioned embodiment is a tampon for pressure on the entire inner surface of the nose and is not suitable for use in nosebleeds.

As a result, due to the above-mentioned drawbacks, and the inadequacy of the available solutions, an improvement in the relevant technical field was required.

### BRIEF DESCRIPTION OF THE INVENTION

The present invention relates to a nose clip that meets the above-mentioned requirements, eliminates all disadvantages and brings some additional advantages.

The primary object of the present invention is to provide a biocompatible hemostatic nose clip which is intended for use in nosebleeds (epistaxis) that originate from the nasal septum, suitable for the personal use of children or adults without the need for medical personnel, insertable or removable with one hand.

A similar object of the invention is to provide a hemostatic nasal clip which can be easily and safely used with one touch without the need for anatomical information by any aged people for emergency intervention in nosebleeds.

Another object of the invention is to provide a hemostatic nose clip in which the user can press directly on the bleeding site with one hand and one movement without the need to determine the bleeding area of the user, thanks to its unique design.

Another object of the invention is to provide a hemostatic nose clip which does not damage the nose or tissues, has no toxic effect and can remain in the nose for up to 10 days, thanks to its biocompatible medical silicone coated structure.

Another object of the invention, thanks to its unique design placed inside the nose and its fine structure that will not affect the nasal breathing, is to provide a hemostatic nose clip which exerts pressure only on Little's area of both sides of the nasal septum, which does not obstruct the nostrils and airway, allows the user to continue breathing through the nose, as the nostrils are open which allows their cleaning by blowing up the clots in the nose or by washing them with water/saline.

Another object of the invention is to provide a hemostatic nose clip which, when placed on the nose, does not close the upper lip of the user, so that the user does not have difficulty feeding, speaking, swallowing for minutes, hours or days, since the retaining portion outside the nose is upward towards the tip of the nose.

Another object of the invention is to provide an inexpensive even disposable hemostatic nose clip suitable for use by anyone.

Another object of the invention is to provide a hemostatic nose clip that does not require any health care professional that adults can easily apply to themselves or to children.

Another object of the invention is to provide a hemostatic nose clip that can be kept in medicine cabinets, first aid kits and emergency response kits in all private and public institutions such as schools, hospitals, land-air-sea vehicles, houses with the simple instructions stated on it; since it does not need to be performed by a qualified medical staff, and it can be produced in any size for adults or children.

Another object of the invention is to provide a hemostatic nose clip which can be used instead of nasal tamponade in anterior epistaxis which cannot be stopped despite interventions such as cauterization in health institutions.

For accomplishing the above-described purposes, the invention is a nose clip suitable for one-hand use for stopping nosebleed, comprising;
at least one body
   remaining outside of the nose during application,
   positioned upwards in the vertical plane on the clip,
   having an open ring structure with open ends passing over each other and running in transverse directions,
   providing the clip to be opened when squeezed from two points and the clip to be closed again when released;
at least two arms
   remaining in nostrils during application,
   connected from their one ends to the open ends of the body,
   positioned mutually at identical levels in the horizontal plane,
   positioned mutually so that the distance between them narrowing,
   having equal lengths covering the distance from the entrance of the nostril entry to the beginning of the Little's area;
at least two hands
   positioned on the Little's area on the nasal septum during application,
   connected to the other ends of the arms in concentric mutually,
   being in contact with each other when the clip is in the closed position,
   having pressure surfaces which provide the necessary pressure to stop the blood flow in the Little's area, when the clip is inserted into the nose.

The structural and characteristic features and all advantages of the invention will be more clearly understood from the following figures and the detailed description written with reference to these figures, and therefore the evaluation should be made with reference to these figures and detailed description.

### BRIEF DESCRIPTION OF FIGURES

FIG. 1 is a side-oblique view of the metal embodiment of the invention when closed.

FIG. 2 is a top-oblique view of the metal embodiment of the invention when closed.

FIG. 3 is an side-oblique view of the metal embodiment of the invention when open.

FIG. 4 is a side view of the metal embodiment of the invention when closed.

FIG. 5 is an top-oblique view of the metal embodiment of the invention when open.

FIG. 6 is a view of the intranasal application of the metal embodiment of the invention.

FIG. 7 is a view of the metal embodiment of the invention in the nose after application.

FIG. 8 is a top-oblique view of the silicone embodiment of the invention when closed.

FIG. 9 is an top-oblique view of the silicone embodiment of the invention when open.

FIG. 10 is a side view of the silicone embodiment of the invention when closed.

FIG. 11 is a front view of the silicone embodiment of the invention when closed.

FIG. 12 is a top-oblique view of the silicone embodiment of the invention when closed.

FIG. 13 is a front view of the silicone embodiment of the invention when open.

FIG. 14 is a top-oblique view of the silicone embodiment of the invention when open.

FIG. 15 is a view of grid structured inner surface of the concave hands of the silicone embodiment of the invention.

The drawings do not necessarily have to be scaled and details which are not necessary to understand the present invention may be omitted. Furthermore, elements that are at least substantially identical or that have at least substantially identical functions are designated by the same number.

### BRIEF DESCRIPTION OF REFERENCES

1 clip

10 body

11, 12 ends

13 holding point

20 arms

30 hands

31 pressure surface

### DETAILED DESCRIPTION OF THE INVENTION

In this detailed description, the nose clip (1) according to the invention is explained only for a better understanding of the subject and with no limiting effect.

The present invention is a hemostatic nasal clip (1) that is suitable for personal use and provides a fast, effective solution to simple nosebleeds encountered in everyday situations. Thanks to its biocompatible structure and alternative structures suitable for one-handed use for users of all ages, it is effective in stopping nosebleeds without requiring health personnel or going to the health institution.

In a basic aspect, the invention comprises a flexible body (10) which allows the clip (1) to be opened when mutual pressure is applied on it with two fingers of the user and returns to its original state when the pressure is removed; arms (20) those connected to the continuation of the body (10) and entering into the nostrils; and hands (30) connected to the arms (20), which provide to stop bleeding by applying pressure to the Little's area on two opposing surfaces of the septum by means of the pressure surfaces (31) on them.

According to the invention the material of the body (10) providing opening and closing the clip (1) is selected from materials which provide a simple spring-like movement character, those flexible enough to allow the open ends (11, 12) of the body (10) passing over each other to move away from each other when the user exerts pressure, and when the pressure is removed, diverged ends (11, 12) are returned to their original position.

The clip (1) comprising body (10), arms (20) and hands (30) is designed in round, square, rectangular or in different forms and in sizes to be suitable for the personal use of children and adults of all ages.

In practice, the user presses the body (10) by holding it with two fingers, and releases the body (10) after placing the opened arms (20) and hands (30) of the clip (1) into the nostrils so that the clip (1) is closed. In order to remove, the user again presses the body (10) with two fingers and opens the clip (1) and pulls it back out of the nose. The body (10) can be connected upwardly in the vertical plane with an appropriate angle to the arms (20) for ease of use.

An important feature of the body (10) structure of the present clip (1), which is different from the present techniques, is that the body (10) is positioned on the clip (1) in a vertical plane upwardly towards the nose tip. Thus, after the clip (1) is placed on the nose, since the body (10) is upward towards the tip of the nose, it does not close the upper lip of the user, and does not interfere with oral functions such as eating, drinking or talking.

The arms (20) contained by the present clip (1) remain in the nostrils during administration. The main function of the arms (20) is to form a connection between the body (10) and the hands (30). The arms (20) are connected at one end to the open ends (11, 12) of the body (10), while the other ends are connected to the hands (30). The arms (20) are mutually connected at the same level in the horizontal plane on the clip (1). There are mutually predetermined distances between the arms (20). The maximum distance between the arms (20) in the region where they are connected to the open ends (11, 12) of the body (10) gradually narrows, reaching a minimum in the region where they are connected to the hands (30). Thus, the portions of the arms (20) remaining in the nose do not exert pressure on the mucosa of the septum and do not damage the tissue. This inwardly sloping embodiment ensures that the arms (20) operate without impacting the nasal mucosa, and that only the hands (30) are pressurized to the septum on the Little's area. The most important feature of the mentioned arms (20) which is different from the present technique is that these arms are of equal length from the nose entry to the beginning of the Little's area. This limits the depth at which they can be inserted into the nostrils. This length is in the range of 1-1.5 cm in clips (1) designed for adults. In alternative embodiments, this length is determined differently for users of all ages. Thus, the user can provide bleeding stopping pressure directly in the Little area when the clip is inserted, without consulting any adjustment or expert medical personnel.

In case of nosebleeds, the arms (20) and the subsequent parts hands (30) of the clip (1) are inserted into the nostrils. The hands (30) are connected to the arms (20) such that they are mutually concentric and evenly level in the horizontal plane. The hands (30) cause the nasal septum to be slightly pressed inward when the clip (1) is in the closed position to contact the Little's area of the nasal mucosa where the nosebleed is generally caused. In this way, the hands (30) which are placed to cover the Little region create mutually opposing pressure on the nose septum. Nasal inhalation and exhalation are not affected as the user's nose airway is open while the hands (30) are closed to create pressure on the septum. Thanks to this optimum pressure provided on the septum, this mechanical effect in the nose stops bleeding and, unlike the present art, no pharmaceutical or chemical adjuvant is needed.

In the context of the present invention, the mentioned clips (1) can be made of biocompatible metal, plastic, silicone or combinations thereof.

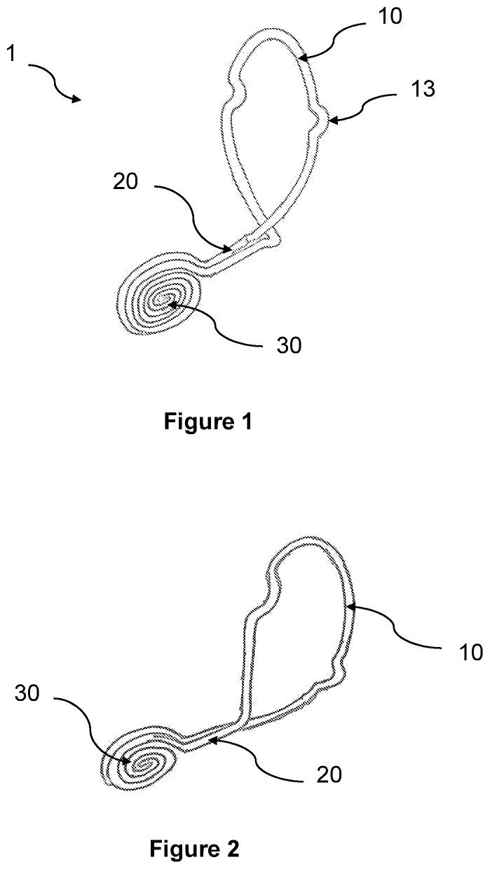

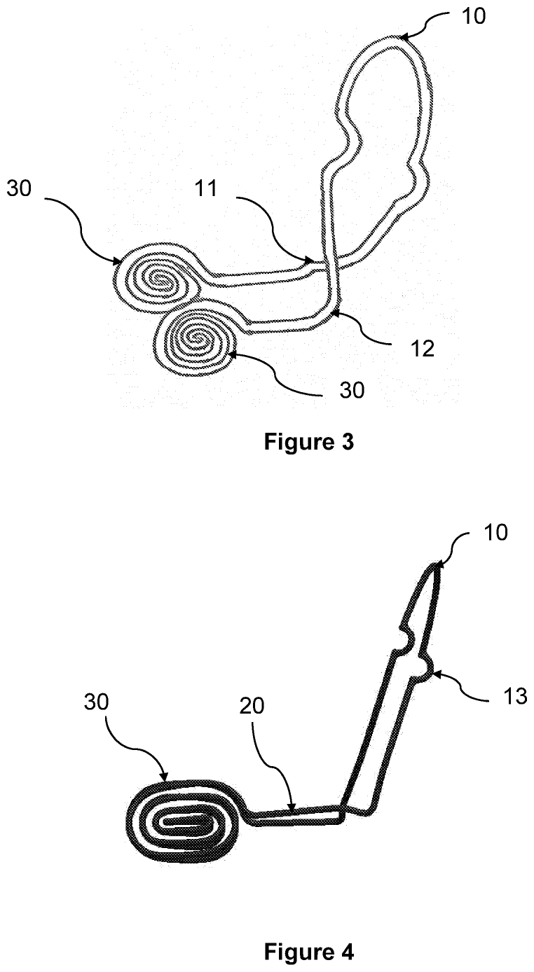

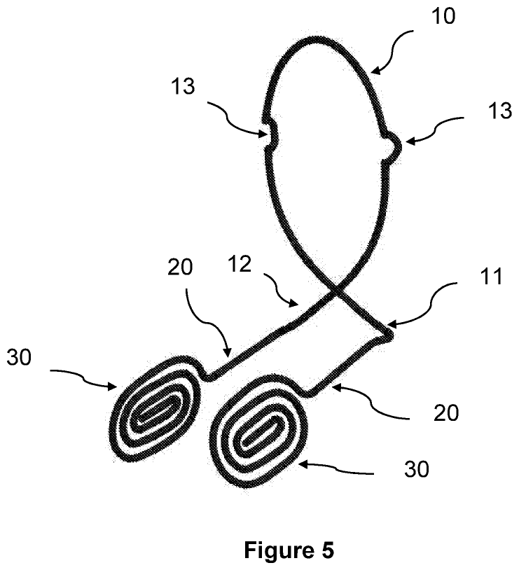

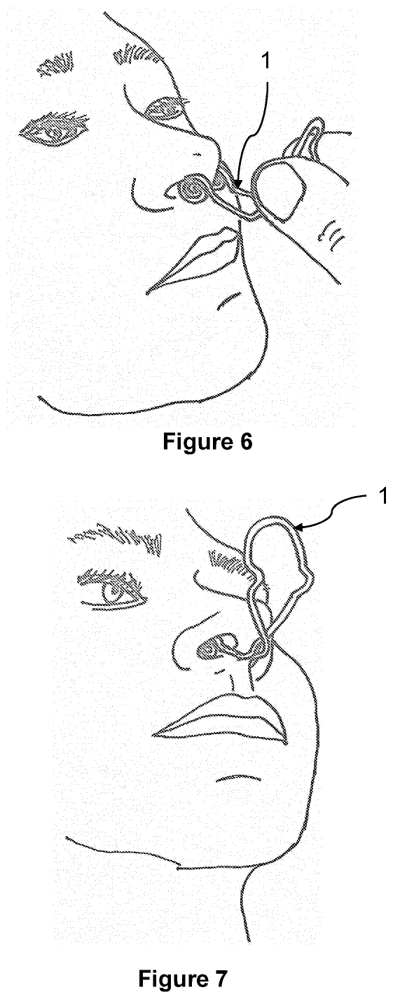

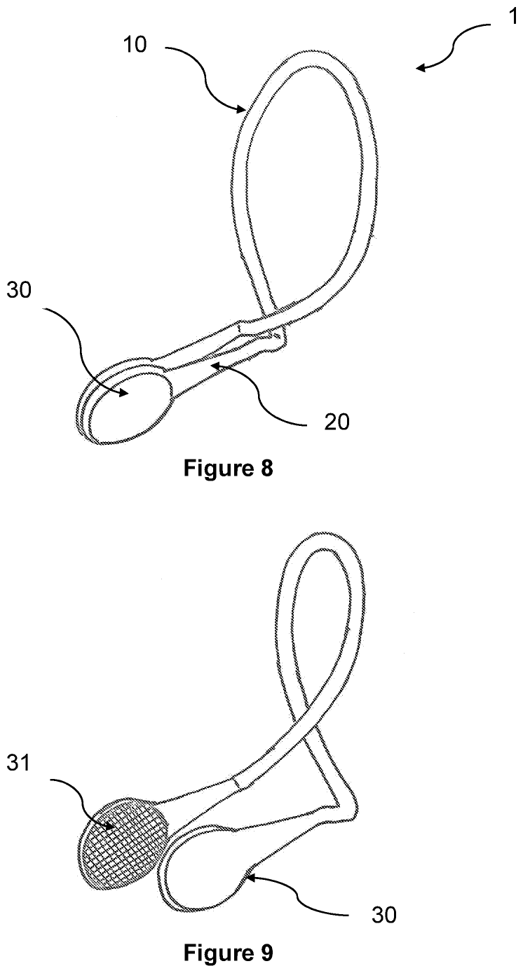

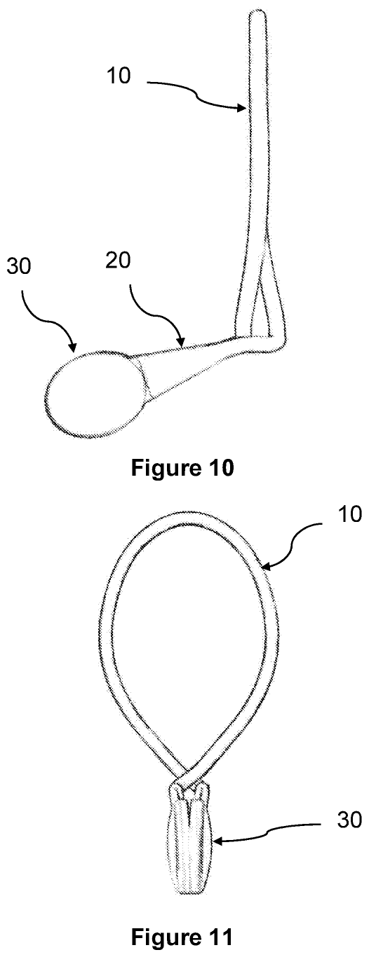

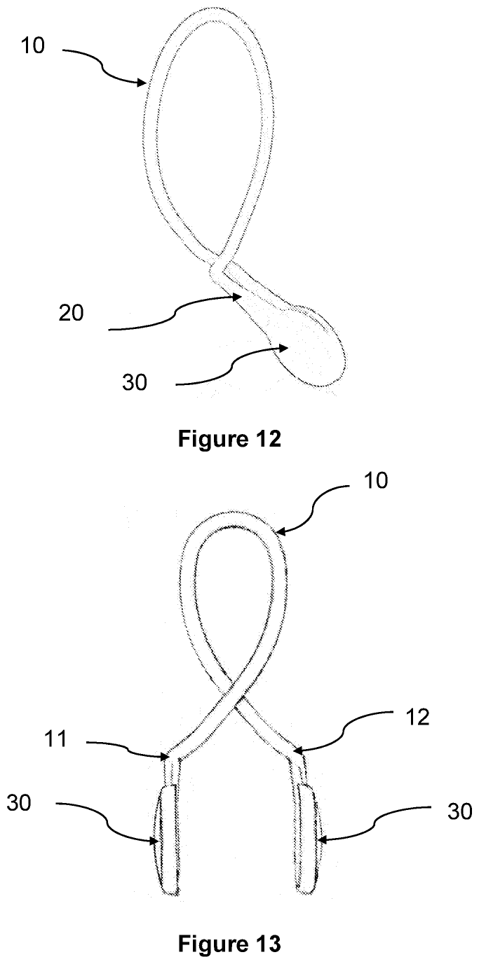

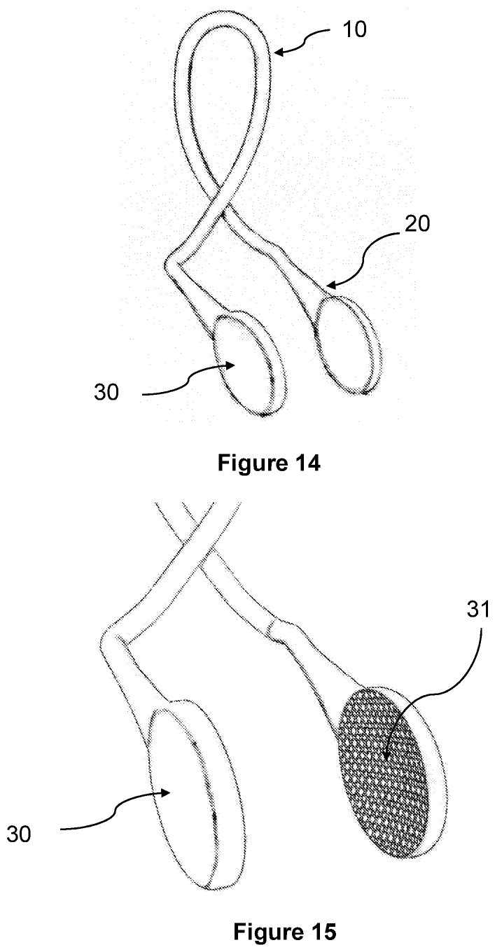

A preferred embodiment of the invention is the medical silicone coated metal clip (1) shown in FIG. 1. In this embodiment, the body (10), arms (20) and hands (30) are produced in a monolithic structure. By means of bending of metal (preferably of flexible steel wire), the body (10), the arms (20), the hands (30) are formed, and a monolithic clip (1) is produced. This produced metal clip (1) is preferably coated with medical silicone by dipping. This provides a biocompatible product suitable for everyone. In this embodiment, furthermore, at least one preferably two opposing points on the body (10) are shaped so that the metal protrudes outwardly, holding points (13) are formed to facilitate opening of the clip (1).

In this embodiment, the body (10) and the arms (20) are formed by a single layer bending of the metal wire; further, the hands (30) are obtained in the form of round or ellipse by bending the continuous metal wire into a spiral. The number of coils here varies depending on the type and dimensional properties of the metal material used and the size of the Little's area of the intended user. When the metal spiral hands (30) obtained by bending are coated with silicone, a recessed-protruded pressure surface (31) is obtained. This pressure surface (31) fully covers the Little's area; thanks to its recessed-protruded structure, it is prevented from sliding from the nasal mucosa. The indented protruding helical structure of the pressure surface (31) allows to exert pressure on all blood vessels from different regions from several different areas in the Little's area. In this embodiment, the hands (30) do not cause nasal congestion in practice since they are formed by bending from a very thin metal wire; it can stand in the nose for a long time and does not prevent nasal washing and blowing during use.

Another preferred embodiment of the invention is the silicone clip (1) embodiment shown in FIG. 2. In this embodiment, the body (10), arms (20) and hands (30) are produced in a monolithic structure by casting method of medical silicone. This provides a biocompatible product suitable for everyone. In this embodiment, the body (10) is preferably round or elliptical.

In this embodiment, the arms (20) in the area to which the body (10) is connected start preferably in a circular cross-section structure ; it continues in a structure that thickens in the horizontal plane until it is connected to the hands (30), and which expands by gradually flattening in the vertical plane. Thus, the clamping force (effect) is transmitted homogeneously to the hands (30).

In this embodiment, the hands (30) may be round or elliptical. The pressing surface (30), which is formed on the hands (30), which is formed on the face facing the nasal septum, is concave. In this way, it can apply pressure on every area it encounters.

This structure also allows the Little area to exert pressure on all blood vessels coming from different areas. The concave surfaces (31) of the hands (30) facing the nasal septum have recessed-protruded structure in the form of a grid. Thus, when the invention is inserted into the nose, it is prevented from slipping through the nasal mucosa. In this embodiment, since the hands (30) are produced in very fine dimensions by casting, they do not cause nasal congestion in practice, can remain in the nose for a long time and do not prevent nasal washing and blowing during use.

In the application of the clip (1) according to the invention, the arms (20) and the hands (30) open outwards (sideways), when the body (10) is squeezed, preferably at the holding points (13). In its open form, it is inserted into both nostrils. When the hands (30) reach the Little's area in front of the nasal septum or the bleeding region, the compression applied to the body (10) is released. In this case, the arms (20) and the hands (30) approach towards each other and evert pressure on the nasal septum (bleeding area). The bleeding is stopped by compressing the bleeding vessel/vessels. 5-10 minutes after the bleeding stops, the body (10) is squeezed again from the holding points (13) and the hands (30) are opened outwards. It's checked whether the bleeding recurs or not. If the bleeding has stopped, the clip (1) is removed from the nose, and if not, the body (10) is released, the hands (30) are closed again and the bleeding site is compressed. The clip (1) of the present invention may remain in the nose for minutes/hours/days with the hands (30) closed. If necessary, the person can go to the health facility. In people with frequent recurrent nosebleeds (allergies, blood diseases, etc.), the invention can be reused after washing and sterilization.

1. A nose clip suitable for one-hand use for stopping a nosebleed, the nose clip comprising:
at least one body remaining outside of the nose during application, the at least one body positioned upwards in a vertical plane on the clip, and having an open ring structure with open ends passing over each other and running in transverse directions, providing the clip to be opened when squeezed from two points and the clip to be closed again when released;
at least two arms remaining in nostrils during application, the at least two arms connected from their one ends to the open ends of the body, the at least two arms positioned mutually at identical levels in the horizontal plane, and positioned mutually so that the distance between them narrows, the at least two arm having equal lengths covering the distance from the nostril entry to the beginning of the Little's area; and
at least two hands positioned on the Little's area on the nasal septum during application, and connected to the other ends of the arms in concentric mutually, the at least two hands being in contact with each other when the clip is in the closed position, and having pressure surfaces which provide the necessary pressure to stop the blood flow in the Little's area, when the clip is inserted into the nose.

2. The nose clip of claim 1, comprising at least one holding point formed on the body.

3. The nose clip of claim 1, comprising a monolithic structure made of medical silicone coated metal wire.

4. The nose clip of claim 3, wherein the hands have a helical recessed-protruded pressure surface.

5. The nose clip of claim 1, comprising a monolithic structure made of medical silicone.

6. The nose clip of claim 5, wherein the hands comprise concave structured hands with grid-shaped recessed-protruded pressure surfaces.
